(19) United States
(12) Patent Application Publication
(10) Pub. No.: US 2022/0226520 A1
(43) Pub. Date: Jul. 21, 2022

(54) GAME MACHINE

(71) Applicant: OMRON CORPORATION, Kyoto-shi (JP)

(72) Inventors: Takuya KUDO, Kyoto-shi (JP); Takehiro AGATA, Kyoto-shi (JP)

(73) Assignee: OMRON CORPORATION, Kyoto-shi (JP)

(21) Appl. No.: 17/555,999

(22) Filed: Dec. 20, 2021

(30) Foreign Application Priority Data
Jan. 15, 2021 (JP) 2021-004737

(51) Int. Cl. A61L 2/10; G07F 17/32; A61L 2/24

(52) U.S. Cl. CPC A61L 2/10; G07F 17/3209; G07F 17/3244; A61L 2202/11; A61L 2/24; A61L 2202/20; A61L 2202/14; G07F 17/3223

(57) ABSTRACT

A game machine (1) comprises a push button (20) that comes out of an opening; an elevating mechanism (51) that raises and lowers the push button (20); a shutter (31) that blocks off the opening in a closed state; a shutter mechanism (32) that opens and closes the shutter; a light irradiation unit (40) having a light source that is disposed on the back surface of the shutter (31), is opposite the operation surface of the push button (20) in the closed state of the shutter (31), and emits a light beam that inactivates a virus; and a control device (70). The control device (70) lowers the push button (20) when an event occurs that triggers a disinfection operation, and turns on the light source for a specific length of time after the shutter (31) has been closed, and puts the shutter (31) in the open state and raises the push button (20) upon completion of the lighting.

### CROSS-REFERENCE TO RELATED APPLICATIONS

This application claims priority to Japanese Patent Application No. 2021-004737 filed on Jan. 15, 2021. The entire disclosure of Japanese Patent Application No. 2021-004737 is hereby incorporated herein by reference.

### BACKGROUND

### Technical Field

The present invention relates to a game machine, and more precisely relates to a game machine having a push button.

### Description of the Related Art

Some game machines installed in casinos, arcades, and other such game facilities are equipped with push buttons (for example, Patent Literature 1). Since these push buttons are operated by many different players, there is the risk that a virus that was clinging to the hand of a player who previously operated the push button will adhere to the operation surface of the push button. Therefore, in order for the next player to be able to play the game without worrying about any adhering virus, the employees of the game facility, etc., have to apply a disinfectant or the like to disinfect the operation surface frequently. In this specification, the word “disinfect” is used to mean “inactivate an adhering virus.” Also, push buttons have also been made of a material containing an antibacterial agent in the past (for example, Patent Literature 2).

### CITATION LIST

### Patent Literature

Patent Literature 1: JP-A 2016-214318

Patent Literature 2: JP-A H9-204560

### SUMMARY

However, frequent disinfection work increases the burden on employees, and disinfection using a disinfectant also has a problem in that it accelerates the deterioration of the push button. On the other hand, a push button made of a material containing an antibacterial agent does not require disinfection work by employees, and therefore does not impose any more work on the facility operator, nor is the push button deteriorated by a disinfectant. However, this approach is not as appealing to users as disinfection work performed by an employee, and the user may become anxious about the instantaneous efficacy of inactivating an adhering virus, or the disinfecting effect of a material containing an antibacterial agent.

It is an object of one mode of the present invention to realize a game machine with which any viruses adhering to an operation surface can be inactivated without relying on manual disinfection work, thereby allowing players to enjoy the game with more peace of mind.

The present invention employs the following configuration in order to solve the above problem.

Specifically, the game machine according to one aspect of the present invention comprises a push button; a panel equipped with an opening through which the push button comes out; an elevating mechanism that raises and lowers the push button; a shutter that blocks off the opening in a closed state; a shutter mechanism that opens and closes the shutter; a light irradiation unit having a light source that is disposed on the back surface of the shutter, is opposite the operation surface of the push button in the closed state of the shutter, and emits a light beam that inactivates a virus; and a control device that controls the elevating mechanism, the shutter mechanism, and the light irradiation unit. The control device lowers the push button when an event occurs that triggers a disinfection operation for inactivating a virus adhering to the operation surface with the light beam, and turns on the light source for a specific length of time after the shutter has been closed, and puts the shutter in an open state and raises the push button upon completion of a lighting.

With the above configuration, the push button is raised and lowered by the elevating mechanism, and comes out of the opening formed in the panel at raised and lowered positions. The shutter is opened and closed by the shutter mechanism, and blocks off the opening formed in the panel in its closed state. The light irradiation unit is disposed on the back surface of the shutter. The light irradiation unit comprises a light source that emits a light beam that inactivates a virus, and is provided so as to be opposite the operation surface of the push button when the shutter is closed. The control device moves the push button to the lowered position when an event occurs that triggers a disinfection operation, puts the shutter in its closed state when the push button moves to the lowered position, and turns on the light source for a specific length of time when the shutter is closed.

Consequently, any virus adhering to the operation surface can be inactivated (killed) by the light beam emitted from the light irradiation unit, without relying on disinfection work performed manually. Also, since the light beam is emitted in a state in which the push button is covered by the shutter, the light rays can be prevented from leaking to the outside. Moreover, because an elevating mechanism is provided to raise and lower the push button, the shutter can have a flat configuration that slides on the back surface of the panel, and this takes up less installation space than a configuration that has a three-dimensional shape covering the push button.

In the push game machine according to the above-mentioned aspect, the configuration may be such that the event that triggers the execution of the disinfection operation is an operation executed by the game machine at the end of a game played on that game machine. With this configuration, disinfection of the operation surface can be executed by using the operation performed by the game machine at the end of a game as a trigger.

In the push game machine according to the above-mentioned aspect, the configuration may be such that the operation executed by the game machine at the end of the game is a ticket return operation. With this configuration, disinfection of the operation surface can be executed by using the return of a ticket (voucher ticket) discharged to the player at the end of the game as a trigger.

In the push game machine according to the above-mentioned aspect, the configuration may be such that the event that triggers the execution of the disinfection operation is an operation executed by a player of the game machine in order to end the game on that game machine. With this configuration, disinfection of the operation surface is executed by using an operation on the game machine performed by the player in order to end the game at the conclusion of play, such as an operation requesting the return of a voucher ticket, as a trigger.

In the push game machine according to the above-mentioned aspect, the configuration may be such that the control device puts the shutter in its open state and raises the push button immediately after the lighting of the light source is finished. With this configuration, the push button can be brought out immediately after the disinfection is completed, and put in a standby state.

In the push game machine according to the above-mentioned aspect, the configuration may be such that when an event occurs that triggers the coming out of the push button after the lighting of the light source is finished, the control device puts the shutter in its open state and raises the push button.

With the above configuration, after the disinfection is complete, the push button is held in its housed state, and is in a standby state until an event occurs that triggers the coming out of the push button, and the push button comes out. This allows the next player of the game machine to clearly recognize that the push button is clean, having been disinfected and not touched by anyone since.

In the push game machine according to the above-mentioned aspect, the configuration may be such that the event that triggers the coming out of the push button is an operation executed by the player on the game machine in order to start a game on that game machine. With this configuration, a disinfected push button comes out after the performance of an operation by the player on the game machine in order to start the game, such as using the insertion of a banknote or a ticket as a trigger.

In the push game machine according to the above-mentioned mode, the configuration may be such that the elevating mechanism comprises a motor and a pantograph mechanism that moves a base on which the push button is mounted between a lowered position and a raised position by means of the drive force of the motor. With this configuration, using a pantograph mechanism increases the reduction ratio of the gear that transmits the drive force of the motor to the pantograph mechanism, so the push button can be held in the raised position even if a strong force is exerted on the push button coming out of the opening.

In the push game machine according to one aspect, the configuration may comprise an instruction receiving unit that receives, at an arbitrary timing, an instruction to execute a disinfection operation to inactivate a virus adhering to the operation surface with the light beam. With this configuration, the provision of the instruction receiving unit makes it possible to disinfect the operation surface of the push button at an arbitrary timing.

(EFFECTS)

With one mode of the present invention, it is possible to realize a game machine with which any virus adhering to the operation surface can be inactivated without relying on manual disinfection work, so the game can be enjoyed with more peace of mind.

### BRIEF DESCRIPTION OF THE DRAWINGS

FIG. 1 is an oblique view of a game machine according to an embodiment, showing a retracted state in which a push button has been retracted, and a normal state in which the push button has come out;

FIG. 2 is an oblique view of a push button unit installed in the game machine according to an embodiment, showing the normal state in which the push button has come out, and the retracted state in which the push button has been retracted;

FIG. 3 is an oblique view showing the inside of a front case in the push button unit installed in the game machine according to an embodiment, showing a state in which a shutter is open;

FIG. 4 is an oblique view showing the inside of the front case of the push button unit installed in the game machine according to an embodiment, showing a state in which the shutter is closed;

FIG. 5 is an oblique view showing the inside of the back case of the push button unit installed in the game machine according to an embodiment, showing a lowered state in which the push button is in its lowered position;

FIG. 6 is an oblique view showing the inside of the back case of the push button unit installed in the game machine according to an embodiment, showing a raised state in which the push button is in its raised position;

FIG. 7 is a detail view of the main components of the push button unit installed in the game machine according to an embodiment, at the portion where the left and right shutters abut against each other;

FIG. 8 is a schematic view showing the attachment of a timing belt and left and right sliders provided to a shutter mechanism in the push button unit installed in the game machine according to an embodiment;

FIG. 9 is an exploded oblique view of the push button in the push button unit installed in the game machine according to an embodiment;

FIG. 10 is a cross-sectional view of the main components, showing the lowered state and the raised state of a pantograph mechanism provided to an elevating mechanism in the push button unit installed in the game machine according to an embodiment;

FIG. 11 is an explanatory diagram showing the sensing of the position of the push button by third and fourth photosensors in the push button unit installed in the game machine according to an embodiment;

FIG. 12 is a block diagram showing a control system of the game machine according to an embodiment;

FIG. 13 is a flowchart of the main sequence of the disinfection operation in the push button unit installed in the game machine according to an embodiment;

FIG. 14 is a flowchart showing the descent control sequence of the elevating mechanism in the push button unit installed in the game machine according to an embodiment;

FIG. 15 is a flowchart showing the control sequence of the shutter unit in the push button unit installed in the game machine according to an embodiment; and

FIG. 16 is a flowchart showing the main sequence of the disinfection operation in a push button unit 10 installed in the game machine according to a modification example.

### DETAILED DESCRIPTION

An embodiment relating to one aspect of the present disclosure (hereinafter, also referred to as “this embodiment”) will be described with reference to the drawings. In this embodiment, a game machine such as a slot machine is given as an example of one mode of the game machine disclosed herein.

### (1) Application Example

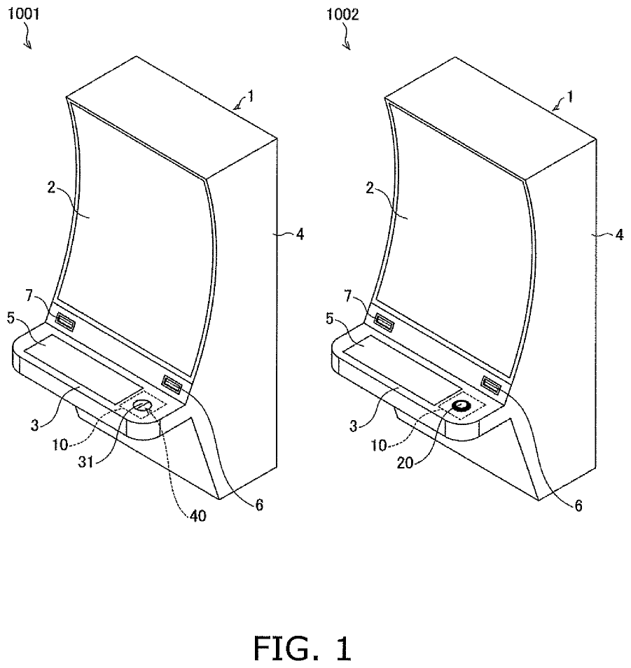

First, an example of a situation in which a game machine 1 is applied will be described with reference to FIG. 1. FIG. 1 is an oblique view of the game machine 1 in this embodiment, and shows a retracted state in which the push button 20 has been retracted, and a normal state in which the push button 20 has come out. 1001 in the drawing shows the retracted state, and 1002 shows the normal state.

As shown in FIG. 1, the game machine 1 comprises an operation panel 5 and a push button 20 on an operation unit 3 located below a main display 2. In the game machine 1, the push button 20 is a button that is always operated in a game. For example, if the game executed by the game machine 1 is a slot machine game, the push button 20 is a spin button that begins the spin. Therefore, the push button 20 is touched much more frequently by the player than the operation panel 5, and the operation surface 20a of the push button 20 is a place where viruses and the like are most likely to adhere.

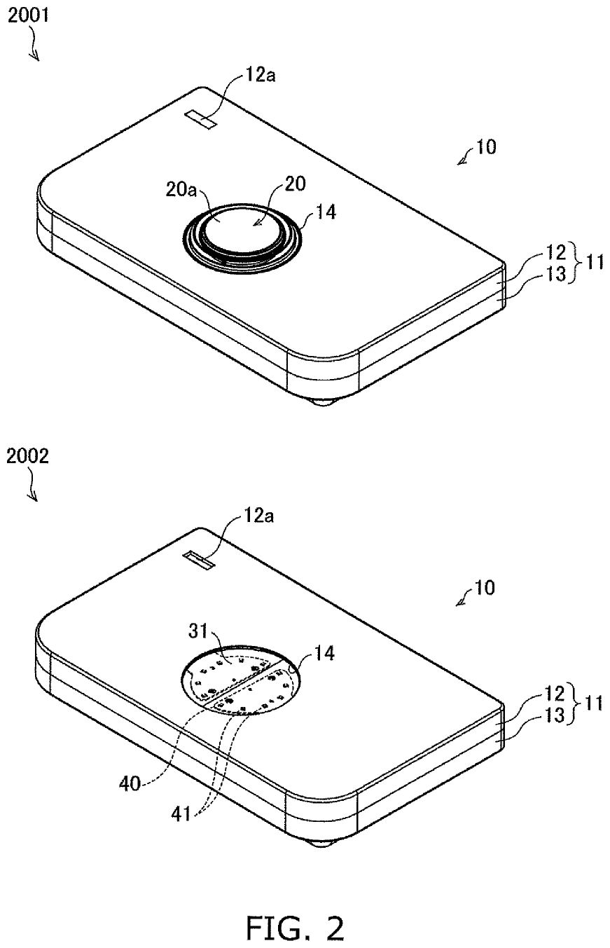

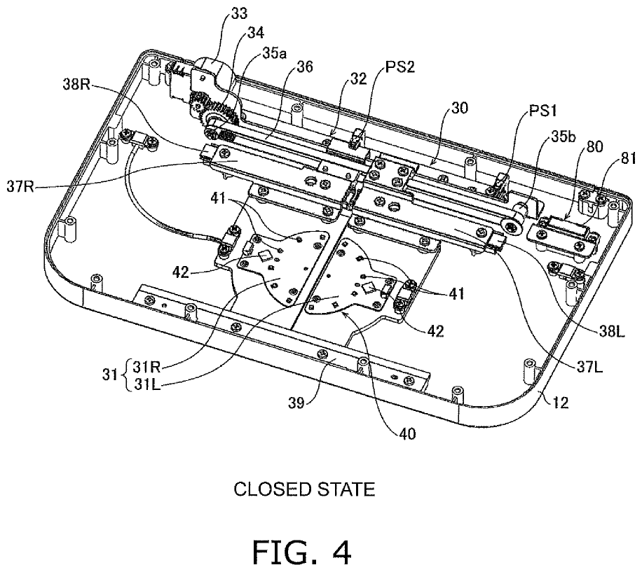

The game machine 1 disclosed herein has a function of disinfecting the operation surface 20a of the push button 20. More specifically, as shown by 2002 in FIG. 2, the push button unit 10 comprising the push button 20 is designed such that the push button 20 retracts into a case 11, and an opening 14 is closed off by a shutter 31. As shown in FIG. 4, disinfecting LEDs 41 are disposed on the back surface of the shutter 31 and ultraviolet rays are emitted from the disinfecting LEDs 41 to inactivate any virus adhering to the operation surface 20a of the push button 20.

This frees employees from having to perform frequent disinfection work, and reduces the burden on the employees. Also, since no disinfectant solution, the operation surface 20a is not deteriorated by a disinfectant solution, and the service life of the push button 20 can be extended. Also, if the player is made aware that the operation surface 20a is disinfected in its retracted state, the player can confirm that the push button 20 has been disinfected whenever the retracted state is seen. Consequently, the notion of the button having been disinfected is more appealing to the player than a push button made of a material containing an antibacterial agent, and the player can rest assured that any virus has been inactivated.

### (2) Configuration Example

### 1. Overview of Game Machine 1

As shown in FIG. 1, the game machine 1 is provided with a main display 2 at a position corresponding to the player's line of sight at the upper part of a housing 4, and the operation unit 3 is provided below the main display 2. On the operation unit 3 is disposed an operation panel 5 constituted by an LCD and a touch panel, and a push button unit 10 comprising a push button 20. Also, a slot 6 for inserting tickets or banknotes, and a payout slot 7 for paying out tickets are provided between the main display 2 and the operation unit 3. Tickets and banknotes inserted from the slot 6 are sent to a ticket/banknote processing unit 8 (discussed below). Also, tickets paid out from a ticket payout unit 9 are discharged from the payout slot 7.

### 2. Overview of Push Button Unit 10

FIG. 2 is an oblique view of the push button unit 10 installed in the game machine 1 according to an embodiment, and shows a normal state in which the push button 20 has come out, and a retracted state in which the push button 20 has been retracted. 2001 in the drawing shows the normal state, and 2002 shows the retracted state.

As shown in FIG. 2, the push button unit 10 comprises a push button 20 and a case 11 for accommodating the push button 20. The case 11 is in the form of a box that is thinner in the pressing direction of the push button 20. The case 11 includes a front case 12 on which the operation surface of the push button 20 is located, and a back case 13 on which the push button 20 is installed. The push button unit 10 is installed in the operation unit 3 such that the upper surface of the front case 12 is flush with the a vanity panel surrounding the operation panel 5, for example (see FIG. 1). The upper surface of the front case 12 corresponds to a panel of the push button unit 10 in which is formed an opening 14 through which the push button 20 comes out.

In the case 11 is formed an opening 14 through which the push button 20 comes out. As shown by 2002 in the drawing, in the normal state the push button 20 has come out of the opening 14, and its operation surface 20a sticks out from the opening 14. On the other hand, as shown by 2002, in the retracted state the push button 20 has gone inside the case 11, and the opening 14 is closed off by the shutter 31.

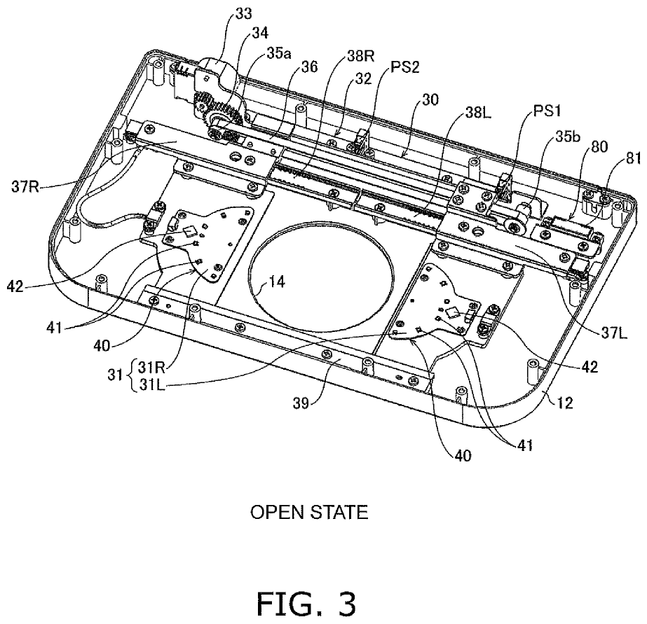

FIG. 3 is an oblique view showing the inside of the front case 12 of the push button unit 10 installed in the game machine 1 according to an embodiment, and shows a state in which the shutter 31 is open. FIG. 4 is an oblique view showing the inside of the front case 12 of the push button unit 10 installed in the game machine 1 according to an embodiment, and shows a state in which the shutter 31 is closed.

As shown in FIGS. 3 and 4, a shutter unit 30 and a sensor unit 80 are installed on the inside (back side) of the front case 12. The shutter unit 30 comprises a shutter 31 that closes off the opening 14, a shutter mechanism 32 that opens and closes the shutter 31, and an ultraviolet irradiation unit (light beam irradiation unit) 40 that is installed on the back surface of the shutter 31. As will be described in detail below, the ultraviolet irradiation unit 40 emits ultraviolet rays as a light beam for inactivating (killing) any virus adhering to the operation surface 20a of the push button 20. The sensor unit 80 receives an instruction from the outside to execute disinfection of the operation surface 20a with the ultraviolet irradiation unit 40.

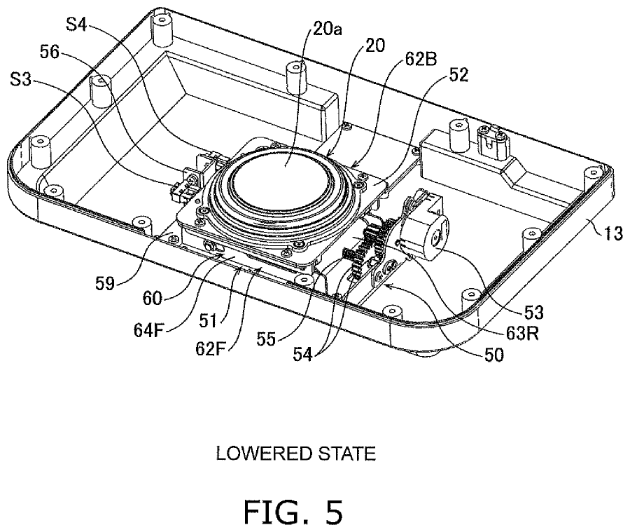

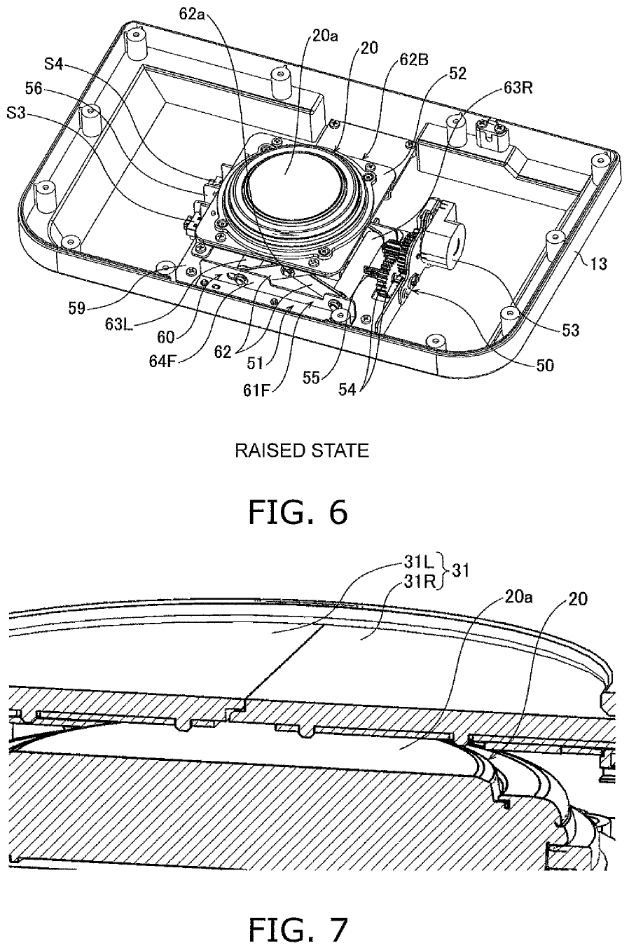

FIG. 5 is an oblique view showing the inside of the back case 13 of the push button unit 10 installed in the game machine 1 according to an embodiment, and shows a lowered state in which the push button 20 is in its lowered position. FIG. 6 is an oblique view showing the inside of the back case 13 of the push button unit 10 installed in the game machine 1 according to an embodiment, and shows a raised state in which the push button 20 is in its raised position.

As shown in FIGS. 5 and 6, an elevating unit 50 is installed on the inside (back side) of the back case 13. The elevating unit 50 is provided with the push button 20 and an elevating mechanism 51 for raising and lowering the push button 20.

### 3. Configuration of Shutter Unit 30

First, the configuration of the shutter unit 30 will be described. As mentioned above, the shutter unit 30 comprises the shutter 31, the shutter mechanism 32, and the ultraviolet irradiation unit 40. These members constituting the shutter mechanism 32 are unitized.

### 3-1. Shutter 31 and Shutter Mechanism 32

As shown in FIGS. 3 and 4, the shutter 31 is provided slidably on the back surface of the front case 12. In this embodiment, the shutter 31 consists of two shutters that move in opposite directions at the same time. For convenience of convenience in the following description, the push button unit 10 shall be assumed to be installed in the operation unit 3 such that the two-piece shutter 31 opens in the left-right direction and the sensor unit 80 is located to the rear. The shutter located on the left side when installed in the game machine 1 is referred to as the left shutter 31L, and the shutter located on the right side is referred to as the right shutter 31R. However, when it is not necessary to discuss the left and right shutters 31L and 31R separately, they shall be referred to simply as the shutter 31.

The left and right shutters 31L and 31R are opened and closed by the shutter mechanism 32, moving closer together during the transition from the open state to the closed state, and moving away from each other during the transition from the closed state to the open state. FIG. 7 is a detail view of the main components of the push button unit 10 installed in the game machine 1 according to an embodiment, at the portion where the left and right shutters 31L and 31R abut against each other. As shown in FIG. 7, the ends of the left and right shutters 31L and 31R that abut each other form a lap joint so as to overlap each other in plan view. This reliably prevents ultraviolet rays from the ultraviolet irradiation unit 40 (discussed below) from leaking out through the gap between the left and right shutters 31L and 31R in the closed state.

As shown in FIGS. 3 and 4, the shutter mechanism 32 comprises an opening and closing motor 33, a reduction gear 34, a pair of pulleys 35a and 35b, a timing belt 36, left and right sliders 37L and 37R, left and right rails 38L and 38R, a shutter rail 39, and first and second photosensors PS1 and PS2.

The opening and closing motor 33 is the drive source for opening and closing the shutter 31. The reduction gear 34 is a component that transmits the drive force of the opening and closing motor 33 to the timing belt 36. The drive shaft 33a of the opening and closing motor 33 engages with the reduction gear 34, and the reduction gear 34 is rotated by the drive force of the opening and closing motor 33. The pulleys 35a and 35b are members that rotate the timing belt 36, and the pulley 35a is fixed to the reduction gear 34. The timing belt 36 is wound around the pulleys 35a and 35b, and teeth formed on the pulley 35a on the drive side mesh with teeth formed on the inside of the timing belt 36.

The left and right sliders 37L and 37R and the left and right rails 38L and 38R are provided corresponding to the left and right shutters 31L and 31R. The left slider 37L is connected to the left shutter 31L and the timing belt 36. When the timing belt 36 rotates, the left slider 37L moves on the left rail 38L, causing the connected left shutter 31L to move. Similarly, the right slider 37R is connected to the right shutter 31R and the timing belt 36. When the timing belt 36 rotates, the right slider 37R moves on the right rail 38R, causing the connected right shutter 31R to move.

The ends of the left and right shutters 31L and 31R that are not connected to the left and right sliders 37L and 37R are supported slidably on the shutter rail 39.

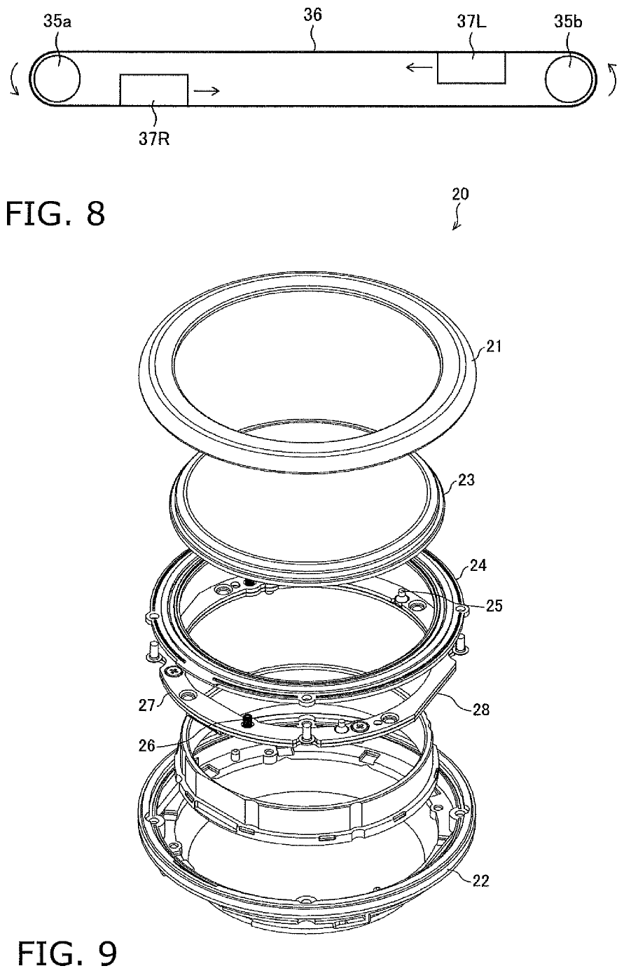

FIG. 8 is a schematic view showing the attachment of the timing belt 36 and the left and right sliders 37L and 37R included in the shutter mechanism 32 of the push button unit 10 installed in the game machine 1 according to an embodiment. As shown in FIG. 8, the left and right sliders 37L and 37R are attached in vertical symmetry at positions of half the circumference of the timing belt 36. Consequently, when the timing belt 36 rotates in the direction indicated by the arrows, the left and right sliders 37L and 37R move closer together, and the left and right shutters 31L and 31R move in the closing direction. Conversely, when the timing belt 36 rotates in the opposite direction from that indicated by the arrows, the left and right sliders 37L and 37R move away from each other, and the left and right shutters 31L and 31R move in the opening direction.

Returning to FIGS. 3 and 4, the first and second photosensors PS1 and PS2 detect whether the shutter 31 is open or closed. The first photosensor PS1 detects an open state (fully open state) in which the shutter 31 is open, and the second photosensor PS2 detects a closed state (fully closed) in which the shutter 31 is closed.

More specifically, the first photosensor PS1 is disposed at a position where light is not blocked until the left shutter 31L is fully opened (see FIG. 4), and where light is blocked by the left slider 37L when the left shutter 31L is fully opened (see FIG. 3). This allows the first photosensor PS1 to detect that the shutter 31 is in its open state when the light is being blocked.

On the other hand, the second photosensor PS2 is disposed at a position where light is not blocked until the right shutter 31R is completely closed (see FIG. 3), and where light is blocked by the right slider 37R when the right shutter 31R is fully closed (see FIG. 4). This allows the second photosensor PS2 to detect that the shutter 31 is in its closed state when the light is being blocked.

As described above, the left and right shutters 31L and 31R move symmetrically as the timing belt 36 rotates, so if the position of either of the left and right shutters 31L and 31R is detected, there is no need to detect the position of the other.

### 3-2. Ultraviolet Irradiation Unit 40

As shown in FIGS. 3 and 4, the ultraviolet irradiation unit 40 is installed on the back surface of the shutter 31 and emits ultraviolet rays as a light beam that inactivates any virus adhering to the operation surface 20a of the push button 20. The ultraviolet irradiation unit 40 comprises disinfecting LEDs (light sources) 41 that emit ultraviolet rays, and an LED drive circuit 42 that drives the disinfecting LED 41. The disinfecting LEDs 41 are disposed so that ultraviolet rays cover the entire operation surface 20a. The disinfecting LEDs 41 and the LED drive circuit 42 are installed on the substrate 43.

In this embodiment, the ultraviolet irradiation unit 40 is divided into two parts that are installed corresponding to the left and right shutters 31L and 31R. When the left and right shutters 31L and 31R are in their closed state, the disinfecting LEDs 41 face the operation surface 20a, and the ultraviolet rays emitted from the disinfecting LEDs 41 irradiate the operation surface 20a.

### 4. Configuration of Elevating Unit 50

Next, the configuration of the elevating unit 50 will be described. As discussed above, the elevating unit 50 comprises the push button 20 and the elevating mechanism 51 for raising and lowering the push button 20.

### 4-1. Push Button 20

FIG. 9 is an exploded oblique view of the push button 20 in the push button unit 10 installed in the game machine 1 according to an embodiment. As shown in FIG. 9, the push button 20 comprises a bezel 21, a base 22, a button body 23, a pressure-sensitive sensor substrate 27, an LED substrate 28, mechanical parts, and so forth. The bezel 21 is a cover member for covering (protecting) the outer peripheral part of the push button 20. The button body 23 is the portion that is pressed, and is supported between the bezel 21 and the base 22 so that the pressing operation can be performed. The upper surface of the button body 23 and the upper surface of the bezel 21 correspond to the operation surface 20a. The button body 23 is made of a transparent material, and an annular rubber portion 24 is fitted to the outer peripheral part thereof. The rubber portion 24 on the outer peripheral part is sandwiched between the bezel 21 and the base 22 so that liquid will not find its way inside the push button 20.

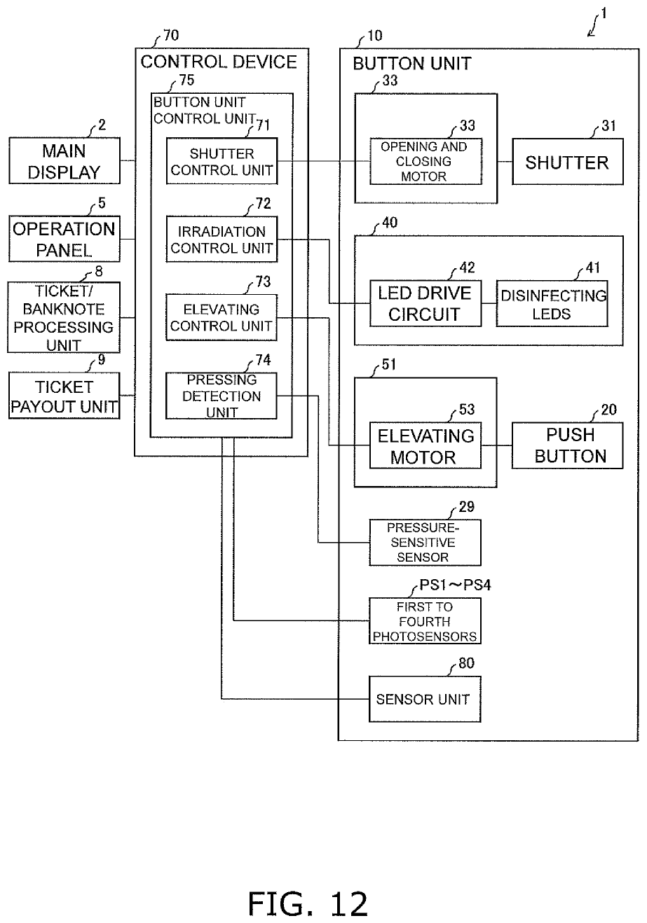

The mechanical parts include a plurality of rubber clickers 25 and a plurality of springs 26. The rubber clickers 25 give a tactile click when the button body 23 is pressed, and the spring 26 returns the pressed button body 23. The pressure-sensitive sensor substrate 27 comprises a pressure-sensitive sensor 29 (see FIG. 12) that detects the pressing of the button body 23, and therefore detects when the button body 23 is pressed. The LED substrate 28 comprises a plurality of LEDs (not shown) and illuminates the outer peripheral portion of the button body 23.

### 4-2. Elevating Mechanism 51

As shown in FIGS. 5 and 6, the push button 20 is installed on a base 52, and is raised and lowered along with the base 52 by the elevating mechanism 51. The elevating mechanism 51 comprises an elevating motor 53, two reduction gears 54, a ball screw 55, a pantograph mechanism 60, and third and fourth photosensors PS3 and PS4. The push button 20 and these members constituting the elevating mechanism 51 are assembled on the base plate to form a unit.

The elevating motor 53 is the drive source that raises and lowers the push button 20. The two reduction gears 54 are components that transmit the drive force of the elevating motor 53 to the ball screw 55. The two reduction gears 54 are rotated by the driving force of the elevating motor 53. One of the reduction gears 54 meshes with the drive shaft (not shown) of the elevating motor 53, and the ball screw 55 is fixed to the shaft of the other reduction gear. The reduction gears 54 are rotated by the drive force of the elevating motor 53. The ball screw 55 is a member that raises and lowers the pantograph mechanism 60.

The pantograph mechanism 60 has a plurality of frame pieces 62 arranged so as to intersect each other, and comprises a frame portion 61 on which an intersection point 62a is axially supported. In this embodiment, the frame portion 61 has two frame pieces 62, and also has a front frame portion 61F and a back frame portion 61B in the front-back direction intersecting the left-right direction, which is the opening and closing direction of the shutter 31.

The upper ends of each of the two frame pieces 62 in the front and back frame portions 61F and 61B are attached to the base 52, and the base 52 is supported by the four frame pieces 62. The lower ends of the two frame pieces 62 in the front and back frame portions 61F and 61B are attached to the shafts of round support columns 63R and 63L disposed on the left and right. More specifically, the lower ends of the two frame pieces 62 of the front frame portion 61F are attached to the front shafts of the left and right round support columns 63L and 63R, and the lower ends of the two frame pieces 62 of the back frame portion 61B are attached to the rear shafts of the left and right round support columns 63L and 63R.

The shafts at both ends of the left and right round support columns 63L and 63R are supported by a front support plate 64F and a back support plate 64B installed in the front-back direction. Of these, the shaft of the right round support column 63R is received at a fixed position by the front and back support plates 64F and 64B. The shaft of the left round support column 63L is slidably received by slots 64a that are formed in the front and back support plates 64F and 64B and are longer in the left-right direction. The ball screw 55 is disposed so as to pass through the center of the left and right round support columns 63R and 63L in the lengthwise direction (front-back direction).

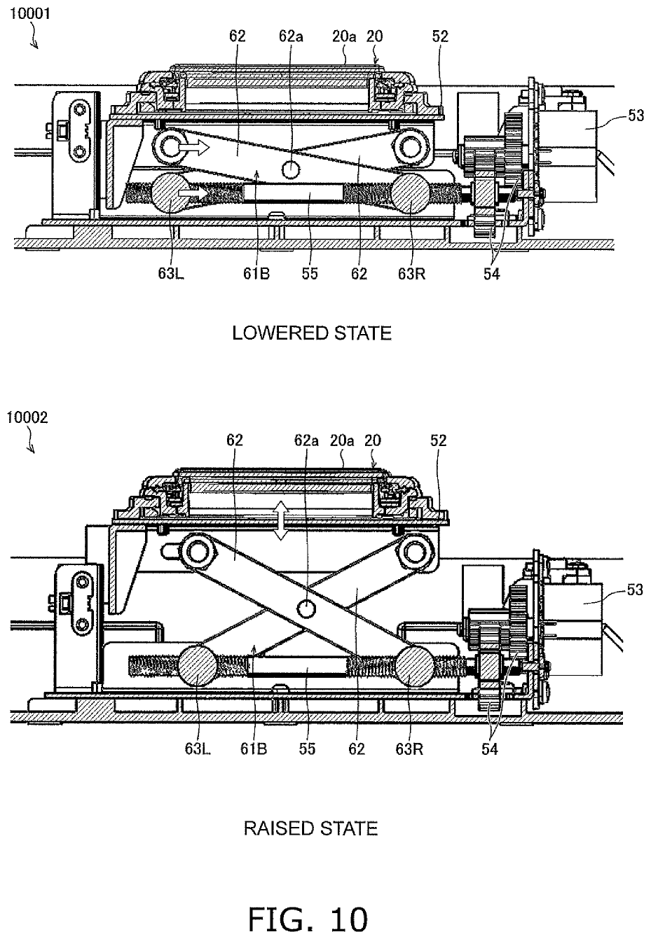

FIG. 10 is a cross-sectional view of the main components, showing the lowered state and the raised state of the pantograph mechanism 60 provided to the elevating mechanism 51 in the push button unit installed in the game machine 1 according to an embodiment. 10001 in the drawing shows the lowered state, and 10002 shows the raised state. As shown by 10001, in the lowered state the left round support column 63L is located at the left end, and the two frame pieces 62 of the front and back frame portions 61F and 61B extend along the ball screw 55. When the ball screw 55 rotates in a specific direction from this state, the left round support column 63L moves horizontally to the right and approaches the right round support column 63R as shown by 10002. When the left round support column 63L approaches the right round support column 63R, the two frame pieces 62 in the front and back frame portions 61F and 61B rise up in the direction away from the ball screw, and the base 52 is pushed up, resulting in the raised state.

When the ball screw 55 rotates from the raised state in the opposite direction from that when the ball screw 55 is raised, the left round support column 63L moves horizontally to the left and away from the right round support column 63R. When the left round support column 63L moves away from the right round support column 63R, the two frame pieces 62 in the front and back frame portions 61F and 61B fold toward the ball screw 55, and the base 52 is lowered, resulting in the lowered state.

In this embodiment, the amount by which the push button 20 is raised by the pantograph mechanism 60 is about 20 mm, and the reduction ratio of the reduction gears 54 is 0.1389. In this case, the elevating motor 53 has to rotate about 15 times to raise the push button 20 by 20 mm Thus increasing the reduction ratio makes the push button 20 more resistant to sinking even if hit forcefully.

Returning to FIGS. 5 and 6, the third and fourth photosensors PS3 and PS4 sense the position of the push button 20. In this embodiment, the third and fourth photosensors PS3 and PS4 are installed on the left side, which is the opposite side from the elevating motor 53. A detection piece 56 that blocks light from reaching the third and fourth photosensors PS3 and PS4 is attached to the base 52.

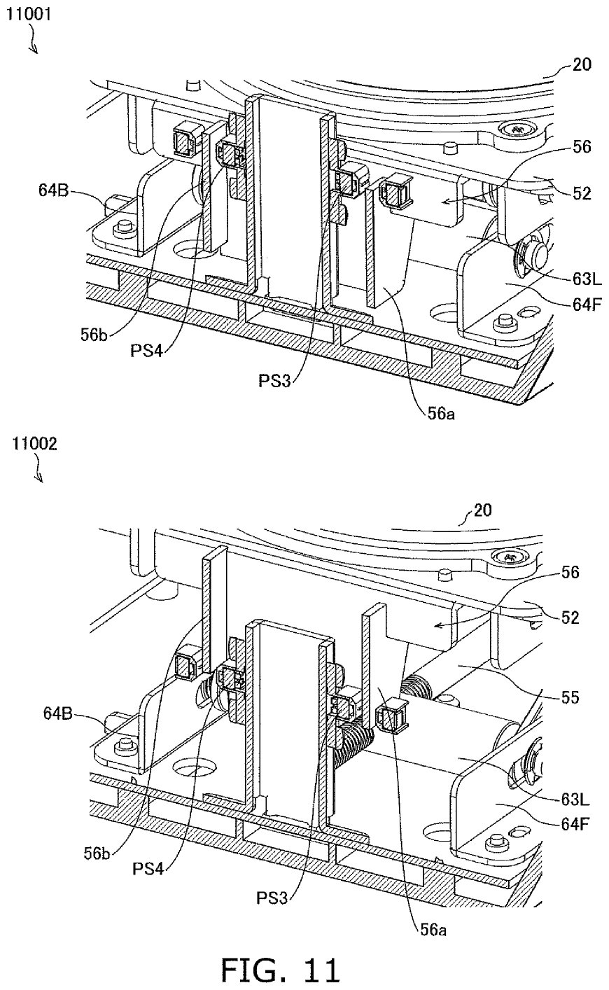

FIG. 11 is an explanatory diagram showing the sensing of the position of the push button 20 by the third and fourth photosensors PS3 and PS4 in the push button unit installed in the game machine 1 according to an embodiment. 11001 in the drawing shows the lowered state (in the lowered position), and 11002 shows the raised state (in the raised position). As shown in FIG. 11, the third and fourth photosensors PS3 and PS4 are installed at the same height, and the detection piece 56 has light blocking walls 56a and 56b that are offset in the up and down direction (height direction).

As by 11001 in the drawing, in the lowered state of the push button 20, the fourth photosensor PS4 is shielded from light by the light blocking wall 56b, and the third photosensor PS3 is not shielded from light by the light blocking wall 56a. When the push button 20 starts to rise, the third photosensor PS3 is also shielded from light by the light blocking wall 56a, and the third and fourth photosensors PS3 and PS4 are both in a light-shielded state.

Then, when the push button 20 reaches its raised position, as shown by 11002, the third photosensor PS3 remains shielded from light by the light blocking wall 56a, but the fourth photosensor PS4 is not shielded from light by the light blocking wall 56b. In this way, the position of the push button 20 (lowered position, raised position, or intermediate position) can be sensed from the combination in which the third and fourth photosensors PS3 and PS4 are or are not shielded from light.

### 4-3. Sensor Unit 80

The sensor unit (instruction receiving unit) 80 receives, from the outside of the push button unit 10, an instruction to execute the disinfection of the operation surface 20a with the ultraviolet irradiation unit 40. As shown in FIGS. 3 and 4, the sensor unit 80 comprises a photosensor 81 that is a reflection sensor, and is attached to the back side of the front case 12. As shown in FIG. 2, a window portion 12a through which the light of the photosensor 81 of the sensor unit 80 passes is provided at the site in the front case 12 where the sensor unit 80 is installed. The player can instruct the execution of the disinfection of the operation surface 20a in a non-contact manner by holding his or her hand over the window portion 12a.

### 5. Control Device 70

FIG. 12 is a block diagram showing a control system of the game machine according to an embodiment. The control device 70 is composed of, for example, a computer device made up of a CPU (central processing unit), an arithmetic processing unit such as a dedicated processor, and the like, and controls the operation of the various components of the game machine 1. The control device 70 is connected to the main display 2, the operation panel 5, the ticket/banknote processing unit 8, the ticket payout unit 9, the push button unit 10, and the like, as the various components of the game machine 1.

The main display 2 displays game images, and the operation panel 5 accepts game selection and various settings performed by the game machine 1. The ticket/banknote processing unit 8 receives tickets or banknotes for playing a game on the game machine 1.

The player can start a game by inserting a ticket or banknote into the ticket/banknote processing unit 8. The ticket payout unit 9 pays out (discharges) a voucher ticket or the like at the end of the game on the game machine 1. The operation of paying out a voucher ticket or the like corresponds to an operation executed by the game machine 1 at the end of the game in the game machine 1.

The push button 20 of the push button unit 10 receives instructions for the selected game that is displayed on the main display 2. As mentioned above, if the game is a slot machine game, this will be a spin button that starts the rotation of the drum. When the push button 20 is operated, the drum displayed on the main display 2 starts to rotate.

In the control device 70, a button unit control unit 75 is constructed in order to control the operation of this push button unit 10. The button unit control unit 75 includes a pressing detection unit 74, a shutter control unit 71, an irradiation control unit 72, and an elevating control unit 73, and is connected to the opening and closing motor 33, the LED drive circuit 42, and the elevating motor 53 in the push button unit 10.

Also, detection signals are inputted to the button unit control unit 75 from the pressure-sensitive sensor 29, the first to fourth photosensors PS1 to S4, and the sensor unit 80. Furthermore, a signal notifying that a ticket has been paid out from the ticket payout unit 9 is inputted to the button unit control unit 75.

The pressing detection unit 74 determines that the push button 20 has been pressed when a detection signal is inputted from the pressure-sensitive sensor 29. When it is determined that the push button 20 has been pressed, the control device 70 starts the rotation of the drum (in the case of a slot machine game).

When an event occurs that triggers a disinfection operation by the ultraviolet irradiation unit 40, the button unit control unit 75 moves the push button 20 to its lowered position, and controls the drive of the opening and closing motor 33, the LED drive circuit 42, and the elevating motor 53 to execute disinfection work. In this embodiment, the button unit control unit 75 determines that an event that triggers a disinfection operation by the ultraviolet irradiation unit 40 has occurred when a signal notifying that a ticket has been paid out is inputted from the ticket payout unit 9.

More precisely, the shutter control unit 71 uses the detection signals of the first and second photosensors PS1 and PS2 to control the drive of the opening and closing motor 33 and to open and close the shutter 31. The irradiation control unit 72 controls the drive of the LED drive circuit 42 to turn on the disinfecting LEDs 41. The elevating control unit 73 uses the detection signals of the third and fourth photosensors PS3 and PS4 to control the drive of the elevating motor 53 and to raise and lower the push button 20.

### 6. Disinfection Operation of Push Button Unit 10

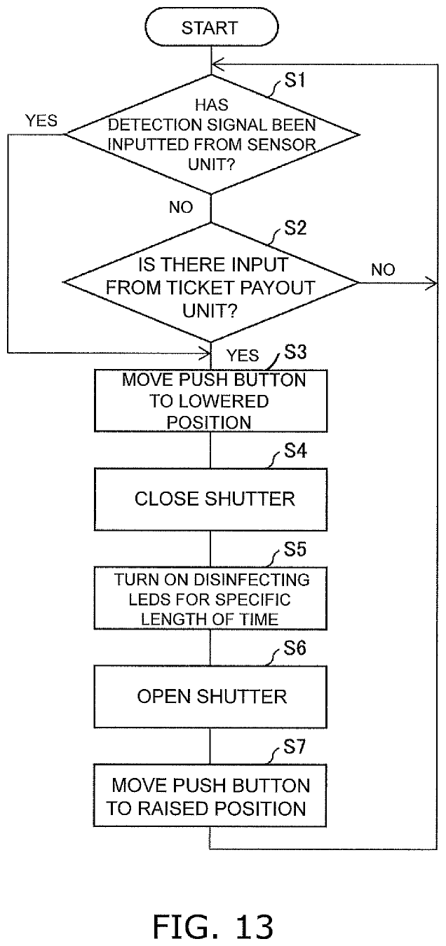

6-1. Main Sequence of Disinfection Operation FIG. 13 is a flowchart of the main sequence of the disinfection operation in the push button unit 10 installed in the game machine 1 according to an embodiment. As shown in FIG. 13, the control device 70 constantly determines whether or not a detection signal has been inputted from the sensor unit 80 and whether or not a detection signal has been inputted from the ticket payout unit 9 (51 and S2). If it is determined in S1 or S2 that a detection signal has been inputted, the elevating control unit 73 drives the elevating motor 53 to move the push button 20 to its lowered position (S3). When the push button 20 moves to its lowered position, the shutter control unit 71 drives the opening and closing motor 33 to close the shutter 31 (S4).

When the shutter 31 closes, the irradiation control unit 72 drives the LED drive circuit 42 to turn on the disinfecting LEDs for a specific length of time (S5). This length of time is long enough for any virus adhering to the operation surface 20a to be inactivated by the ultraviolet rays from the disinfecting LEDs 41, and is set to about 10 seconds, for example. In S4, once the LEDs have been on for the specific length of time, the shutter control unit 71 drives the opening and closing motor 33 to open up the shutter 31 (S6). When the shutter 31 is opened in S6, the elevating control unit 73 drives the elevating motor 53 to move the push button 20 to its raised position (S7). After this, the control device 70 returns to the processing of S1.

### 6-2. Elevating Mechanism Control Sequence

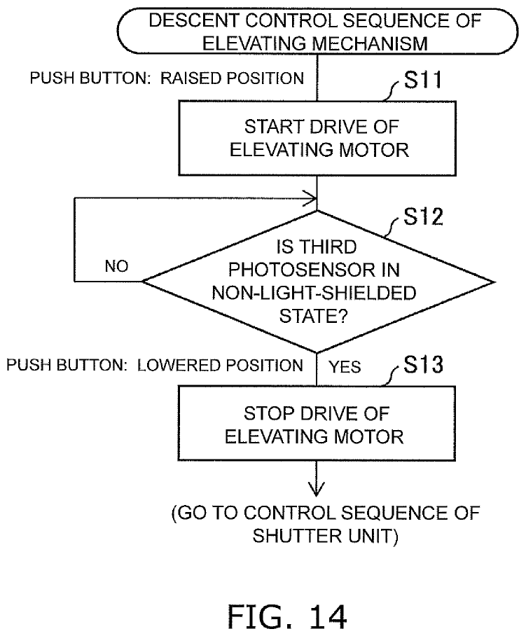

FIG. 14 is a flowchart showing the descent control sequence of the elevating mechanism 51 in the push button unit 10 installed in the game machine 1 according to an embodiment. The descent control sequence of FIG. 14 is executed in S3 of FIG. 13.

As shown by 11002 in FIG. 11, in the raised state in which the push button 20 is in its raised position, the third photosensor PS3 is in a light-shielded state and the fourth photosensor PS4 is in a non-light-shielded state. When the elevating control unit 73 starts driving the elevating motor 53 (S11) in order to lower the push button 20 from its raised position, both the third and fourth photosensors PS3 and PS4 are in a light-shielded state.

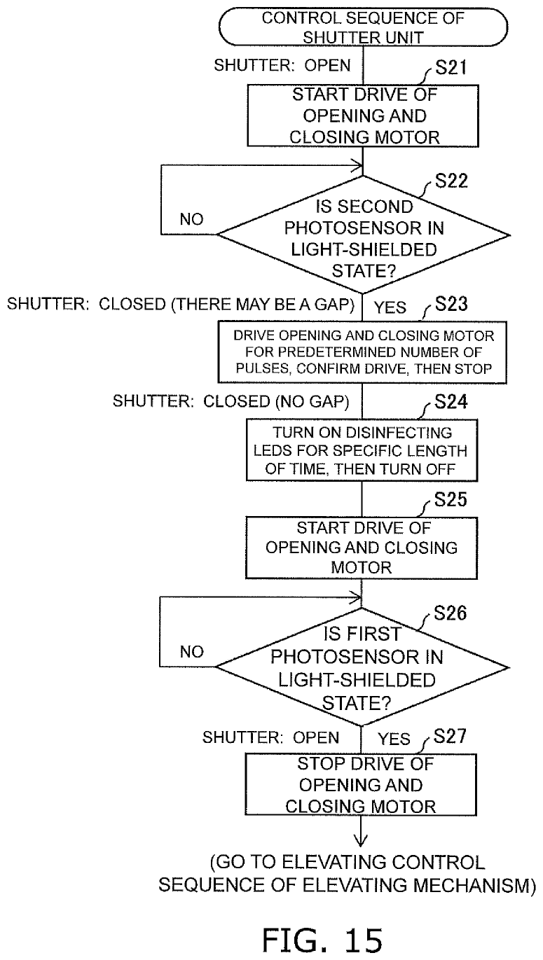

The elevating control unit 73 drives the elevating motor 53 until the third photosensor PS3 is in a non-light-shielded state. As shown by 11001 in FIG. 11, when the push button 20 reaches its lowered position, the third photosensor PS3 is in a non-light-shielded state and the fourth photosensor PS4 is in a light-shielded state. When the third photosensor PS3 is in a non-light-shielded state (Yes in S12), the elevating control unit 73 stops driving the elevating motor 53 (S13). After this, the control device 70 proceeds to the control sequence of the shutter unit 30 shown in FIG. 15.

Although not shown, the ascending control sequence for moving the push button 20 to its raised position, which is executed in S7 of FIG. 13, is the reverse of FIG. 14. That is, the elevating control unit 73 drives the elevating motor 53 to raise the push button 20 until the fourth photosensor PS4 is in a non-light-shielded state, and when the fourth photosensor PS4 is in a non-light-shielded state, the drive of the elevating motor 53 is stopped.

### 6-3. Control Sequence of Shutter Unit 30

FIG. 15 is a flowchart showing the control sequence of the shutter unit 30 in the push button unit 10 installed in the game machine 1 according to an embodiment. The control sequence in FIG. 15 is executed in S4 to S6 of FIG. 13.

As shown in FIG. 3, when the shutter 31 is in its open state (fully open), the first photosensor PS1 is in a light-shielded state and detects this fully open state. The second photosensor PS2 that detects a closed state is in a non-light-shielded state. In order to close the shutter 31, the shutter control unit 71 starts driving the opening and closing motor 33 (S21). When the shutter 31 starts to close, the first and second photosensors PS1 and PS2 are both in a non-light-shielded state. The shutter control unit 71 drives the opening and closing motor 33 until the second photosensor PS2 that detects a closed state is in a light-shielded state. As shown in FIG. 4, when the shutter 31 is closed, the second photosensor PS2 is in a light-shielded state. When the second photosensor is in a light-shielded state (Yes in S22), the shutter control unit 71 drives the opening and closing motor 33 by a specific number of pulses, confirms the drive for this specific number of pulses, and then stops (S23).

This processing of S23 is executed in order to prevent the shutter 31 from not completely closing, which would result in a tiny gap. That is, in the control to stop the opening and closing motor 33 immediately after the second photosensor PS2 is shielded from light by the right slider 37R (Yes in S22), variance in the assembly position of the second photosensor PS2 may produce a tiny gap in the closed state of the shutter 31. If S23 is executed, any variance in the assembly position of the second photosensor PS2 can be absorbed and the shutter 31 can be closed without leaving a tiny gap.

The fact that the shutter 31 is not completely closed can be detected in the following two ways. One is when the second photosensor PS2 is not shielded from light even after a specific period of time has elapsed since the opening and closing motor 33 started to be driven. The other is when it is detected that the opening and closing motor 33 has not rotated by a specific angle even after a specific period of time has elapsed even though the specified number of pulses was sent to the opening and closing motor 33 after the second photosensor PS2 is in a light-shielded state. A Hall element is provided to the motors constituting the opening and closing motor 33 and the like, and this allows the motor to confirm its own rotational position.

When the shutter 31 is completely closed by the processing of S23, the irradiation control unit 72 turns on the disinfecting LEDs 41 for a predetermined length of time and then turns them off (S24). Consequently, the operation surface 20a of the push button 20 is irradiated with ultraviolet rays, and any adhering virus is inactivated.

After confirming that the disinfecting LEDs 41 have been turned off, the shutter control unit 71 starts driving the opening and closing motor 33 in order to open the shutter 31 (S26). When the shutter 31 starts opening, the first and second photosensors PS1 and PS2 both go back into its non-light-shielded state. The shutter control unit 71 drives the opening and closing motor 33 until the first photosensor PS1 that detects an open state is back in its light-shielded state. When the first photosensor PS1 is in its light-shielded state (Yes in S26), the shutter control unit 71 stops driving the opening and closing motor 33 (S27).

### 7. Effect

With the above configuration, the game machine 1 comprises the ultraviolet irradiation unit 40, and in a state in which the push button 20 has been retracted, the operation surface 20a is irradiated with ultraviolet rays to inactivate any virus adhering to the operation surface 20a. This makes it possible to disinfect the operation surface 20a without relying on disinfection work in which a worker applies a disinfectant solution, freeing the worker from frequent disinfection work and reducing the burden on the worker. Also, since no disinfectant solution is used, the operation surface 20a will not be deteriorated by disinfectant solution, and the service life of the push button 20 can be extended.

Also, because a player can recognize that the operation surface 20a has been disinfected in its retracted state, the player can confirm that the push button 20 has been disinfected just by seeing the retracted state. Consequently, the notion of the button having been disinfected is more appealing to the player than a push button made of a material containing an antibacterial agent, and the player can rest assured that any virus has been inactivated.

Also, with the above configuration, the ultraviolet irradiation by the ultraviolet irradiation unit 40 is performed in a state in which the shutter 31 has been completely closed. This reliably prevents the player from looking at the emitted ultraviolet rays.

Also, with the above configuration, the elevating mechanism 51 for raising and lowering the push button 20 is provided, and the shutter 31 closes off the opening 14 in a state in which the push button 20 is lowered. With this configuration, the shutter 31 can have a flat configuration, and the push button unit 10 can be thinner. This allows for installation in a limited space such as the operation unit 3 of the game machine 1 (see FIG. 1).

Also, with the above configuration, the elevating mechanism 51 comprises a pantograph mechanism that raises and lowers the base 52 using the drive force of the elevating motor 53. Using the pantograph mechanism increases the reduction ratio of the reduction gear 54 that transmits the drive force of the elevating motor 53 to the pantograph mechanism, so the push button 20 can be held in its raised position even if a large force is applied to the push button 20.

Also, with the above configuration, since the sensor unit 80 is designed to receive an instruction to execute the disinfection operation at any point in time, the player can disinfect the push button whenever desired. In particular, with the above configuration, since the sensor unit 80 comprises a reflection sensor, a player can issue a command to execute a disinfection operation on the push button in a non-contact manner.

### (3) Modification Examples

An embodiment of the present invention was described in detail above, but the above description is merely an example of the present invention in all respects. It should go without saying that various improvements and modifications are possible without departing from the scope of the present invention. For instance, the following modifications can be made. In the following, Constituent elements that are the same as in the above embodiment will be numbered the same, and points that are the same as in the above embodiment may be omitted as appropriate. The following modification examples can be combined as appropriate.

3.1

In the main sequence of the disinfection operation shown in FIG. 13, the control device 70 (button unit control unit 75) executes S6 and S7 immediately after disinfection with the LEDs 41 for a specific length of time has been completed, and the shutter 31 is released so that the push button 20 comes out. However, the processing of S6 and S7 does not have to come immediately after the completion of lighting of the disinfecting LEDs 41 if these LEDs have already been lit for a specific length of time, and an event that triggers the appearance of the push button 20 may be set in advance.

An example of an event that triggers the appearance of the push button 20 is an operation performed by the player on the game machine 1 in order to start a game on this game machine 1. An example of such an operation is when a ticket or a banknote is inserted into the ticket/banknote processing unit 8 of the game machine 1. Consequently, the push button 20 can be made to come out at the point when the control device 70 has detected that there is a situation (event) in which the push button 20 needs to be operated, such as the start of a game on the game machine 1.

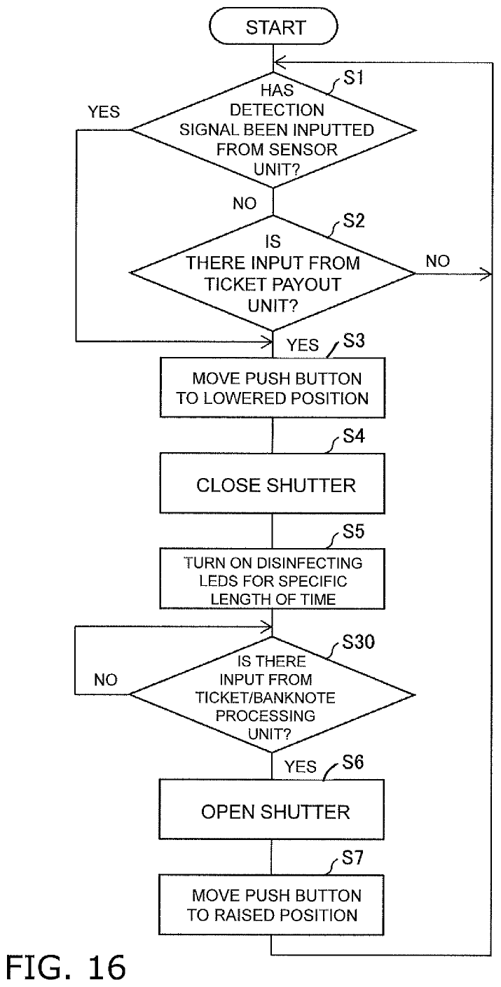

FIG. 16 is a flowchart showing the main sequence of the disinfection operation in the push button unit 10 installed in the game machine according to a modification example. As shown in FIG. 16, the control device 70 performs the processing of S30, in which it is determined whether or not a detection signal has been inputted from the ticket/banknote processing unit 8, in between the processing of S5, in which the disinfecting LED's are turned on for a specific length of time, and the processing of S6 and S7, in which the shutter 31 is opened and the push button 20 comes out. That is, when a detection signal notifying that a ticket or a banknote has been inserted to start a game is inputted from the ticket/banknote processing unit 8 of the game machine 1 (Yes in S30), the shutter 31 is opened and the push button 20 comes out.

With the above configuration, after the disinfection is completed, the push button 20 is in a standby state in which it is held in its retracted state and only comes out when an event occurs that triggers the appearance of the push button 20. Consequently, the next player of the game machine 1 can clearly recognize that the push button 20 is clean, has been disinfected, and has not been touched by anyone since.

3.2

With the above configuration, the push button unit 10 is provided with the sensor unit 80, but the sensor unit 80 is not necessarily needed. Also, when the sensor unit 80 is provided, it may be provided to the back surface of the game machine 1, the back surface of the operation unit 3, or the like.

3.3

With the above configuration, the event that triggers the execution of the disinfection operation by the ultraviolet irradiation unit 40 was the discharge of a voucher ticket, which is discharged to the player at the end of a game on the game machine 1, but may instead be an operation in which the return of a voucher ticket is required, for example. That is, the event that triggers the execution of the disinfection operation by the ultraviolet irradiation unit 40 may be an operation that the player executes on the game machine 1 in order to end a game on the game machine 1. However, if the push button unit 10 is installed in the game machine 1, the event that triggers the execution of the disinfection operation is preferably a player change.

The present invention is not limited to the embodiments given above, and various modifications are possible within the scope of the claims, and any embodiments obtained by suitably combining the technical means disclosed in different embodiments are also encompassed by the technical scope of the present invention.

1. A game machine, comprising:
a push button;
a panel equipped with an opening through which the push button comes out;
an elevating mechanism configured to raise and lower the push button;
a shutter configured to block off the opening in a closed state;
a shutter mechanism configured to open and close the shutter;
a light irradiation unit having a light source that is disposed on a back surface of the shutter, is opposite an operation surface of the push button in the closed state of the shutter, and configured to emit a light beam that inactivates a virus; and
a control device configured to control the elevating mechanism, the shutter mechanism, and the light irradiation unit,
the control device lowers the push button when an event occurs that triggers a disinfection operation for inactivating a virus adhering to the operation surface with the light beam, and turns on the light source for a specific length of time after the shutter has been closed, and puts the shutter in an open state and raises the push button upon completion of a lighting.

2. The game machine according to claim 1, wherein the event that triggers an execution of the disinfection operation is an operation executed by the game machine at an end of a game played on the game machine.

3. The game machine according to claim 2, wherein the operation executed by the game machine at an end of the game is a ticket return operation.

4. The game machine according to claim 1, wherein the event that triggers an execution of the disinfection operation is an operation executed by a player of the game machine in order to end a game on the game machine.

5. The game machine according to claim 1, wherein the control device puts the shutter in the open state and raises the push button immediately after a lighting of the light source is finished.

6. The game machine according to claim 1, wherein when an event occurs that triggers a coming out of the push button after a lighting of the light source is completed, the control device puts the shutter in the open state and raises the push button.

7. The game machine according to claim 6, wherein the event that triggers a coming out of the push button is an operation executed by a player on the game machine in order to start a game on that game machine.

8. The game machine according to claim 1, wherein the elevating mechanism comprises:
a motor; and
a pantograph mechanism configured to move a base on which the push button is mounted between a lowered position and a raised position by means of a drive force of the motor.

9. The game machine according to claim 1, further comprising an instruction receiving unit configured to receive, at an arbitrary timing, an instruction to execute a disinfection operation to inactivate a virus adhering to the operation surface with the light beam.
